(19) United States
(12) Patent Application Publication
(10) Pub. No.: US 2011/0118601 A1
(43) Pub. Date: May 19, 2011

(54) Ultrasound Guided Systems and Methods

(75) Inventors: Darryl E. Barnes, Byron, MN (US); Jay Smith, Byron, MN (US)

(73) Assignee: MAYO FOUNDATION FOR MEDICAL EDUCATION AND RESEARCH NONPROFIT CORPORATION

(21) Appl. No.: 12/918,350

(22) PCT Filed: Feb. 20, 2009
(86) PCT No.: PCT/US09/34688

§ 371 Date: Feb. 8, 2011

Related U.S. Application Data
(60) Provisional application No. 61/030,076, filed on Feb. 20, 2008.
(60) Provisional application No. 61/030,085, filed on Feb. 20, 2008.

(51) Int. Cl. A61B 8/00; A61B 18/00

(52) U.S. Cl. 600/439

(57) ABSTRACT

Coblation probes, systems and methods are described herein, along with tissue dilation apparatus, and kits including both. The articles may be ultrasonically echogenic to assist with guidance.

### RELATED APPLICATIONS

The present application claims the benefit under 35 U.S.C. §119(e) of the following U.S. Provisional Applications: Ser. No. 61/030,076, titled ULTRASOUND COBLATION ARTIFACT REDUCTION SYSTEMS AND METHODS, filed on Feb. 20, 2008 and Ser. No. 61/030,085, titled ULTRASOUND GUIDED TISSUE DILATION APPARATUS, filed on Feb. 20, 2008. Both of these provisional applications are incorporated herein by reference in their entireties.

Tissue coblation apparatus and/or tissue dilation apparatus are described herein that may be used in ultrasonically-guided procedures.

Coblation devices are used to remove tissue from a variety of locations within the body as discussed in, e.g., U.S. Pat. Nos. 6,296,638 (Davison et al.); 5,697,281 (Eggers et al.); and 5,683,366 (Eggers et al.). In some instances, these devices may be used to debride diseased tendons and other soft tissue via open and arthroscopic procedures (using, e.g., the TOPAZ MicroDebrider from ArthroCare Sportsmedicine).

Although coblation devices are useful for the removal of tissue, debridement using Radio-Frequency (RF) energy typically produces gas that forms bubbles. In some instances, the formation of gas bubbles is encouraged. Such gas bubbles, however, have a very high intensity reflection coefficient (IRC) with respect to ultrasonic energy. As a result, coblation devices are not used in ultrasonic guided procedures because the artifacts caused the gas bubbles prevent accurate visualization of the ablation site.

In addition to the issues raised by coblation, a variety of clinical conditions exist in which soft tissues (e.g., nerves, vessels, tendons, etc.) are constricted in the body. Such constrictions can lead to irritation, inflammation, and pain. These conditions are often treated with rest, anti-inflammatory measures (e.g., anti-inflammatory medications, steroid injections, etc.).

In those instances where such treatments are ineffective, surgical release and/or removal of the constricting tissues may be required. Surgical release and/or removal of the constricting tissue, however, typically requires open surgery with the concomitant costs and morbidity.

### SUMMARY

The coblation systems and methods described herein provide for a flow of fluid to the ablation site such that gas bubbles formed during the ablation procedure are removed at a rate that allows for ultrasonic guidance of the procedure. In the systems and methods, fluid is delivered through one or more delivery channels and removed through one or more aspiration channels at the same time. As a result, the fluid preferably moves into and out of the ablation site, with gas bubbles formed as a part of the ablation process being removed with the fluid passing into the one or more aspiration channels. The fluids used may include, e.g., Ringer's lactate solution, and other solutions suitable for use in coblation procedures.

The flow of fluid at the ablation site may advantageously limit collateral tissue damage from the thermal energy generated as a part of the ablation process. In some embodiments, the fluid delivered to the ablation site may be cooled below room temperature (e.g., cooled to a temperature below 20 degrees Celsius). Cooling of the fluid may enhance thermal control at the ablation site because the fluid may be capable of absorbing more thermal energy than warmer fluids delivered to the same site.

Control over the fluid flow to and from the ablation site may be helpful to limit over-infiltration of the soft tissues surrounding the ablation site that may be experienced with conventional fluid delivery systems. That control may include more precise and coordinated control over the fluid delivery, fluid removal, and RF energy by the system.

In addition, the removal of fluid from the ablation site may also result in removal of tissue and/or other waste products.

In some embodiments, the system may include a controller that provides for fluid flow initiation and fluid removal for a selected time period before the delivery of RF energy, with the fluid flow and removal continuing for a selected time period after cessation of the RF energy delivery. For example, one embodiment may provide for fluid delivery/removal initiation at a selected time period of 0.5 seconds or less before RF energy delivery, with the fluid delivery/removal continuing for a period of 1 second or more after the cessation of RF energy delivery. Other selected time periods may be used, with the systems and methods of the present invention.

In addition to the coblation systems and methods, the present disclosure also includes tissue dilation apparatus and methods that may be used in conjunction with the coblation systems or that may be used without the coblation systems. In some embodiments, the tissue dilation apparatus and methods described herein may be used to provide access to and/or relieve non-cardiac stenosis using percutaneous delivery of a tissue dilation device under ultrasound guidance. In use, the tissue dilation apparatus may be passed into areas in which tissue is constricted (by, e.g., scar tissue, adhesions, etc.). The tissue dilation apparatus preferably includes ultrasonically echogenic material such that the position of at least the dilating portion of the apparatus can be determined using ultrasonic guidance. When in a selected position, the expandable portions of the tissue dilation apparatus can be expanded to, e.g., disrupt scar tissue, release adhesions, or otherwise relieve symptomatic stenosis. The tissue dilation apparatus may also include a lumen through which the coblation systems may pass to access targeted body tissue.

Some of the more common clinical conditions that may potentially be treated using the tissue dilation apparatus described herein may include, e.g., stenosing tenosynovitis (e.g., “trigger finger”) and entrapment neuropathy (e.g., carpal tunnel syndrome, radial tunnel syndrome, etc.). In addition to being used for direct therapy, the tissue dilation apparatus described herein may alternatively be used to create working spaces for other ultrasonically guided procedures as described herein. For example, the devices may be used to create a “working space” for other percutaneous procedures. Potential clinical applications may include, but are not limited to, carpal tunnel release, trigger finger release, ultrasonic guided injection or aspiration, delivery of therapeutic agents, etc.

The treatment offered by tissue dilation apparatus described herein may be augmented in some situations by, e.g., therapeutic ultrasonic energy (to, e.g., heat the tissue to be dilated), RF energy, coblation, laser energy, and/or concomitant release of the constriction with, e.g., cutting blades, etc.

In one aspect, a coblation system with ultrasound artifact reduction is provided, the system including a shaft having a proximal end and distal end; one or more delivery lumens extending through the shaft from the proximal end towards the distal end; one or more aspiration lumens extending through the shaft from the proximal end towards the distal end; fluid control apparatus fluidly coupled to the one or more delivery lumens and the one or more aspiration lumens; a coblation probe extending through the shaft, wherein the coblation probe includes a coblation tip proximate the distal end of the shaft; a coblation energy source operatively coupled to the coblation probe to deliver coblation energy to the coblation tip; and a system controller operatively connected to the fluid control apparatus and the coblation energy source, wherein the system controller further: controls operation of the fluid control apparatus such that a fluid is delivered to the distal end of the shaft through the one or more delivery lumens and that at least a portion of the fluid is removed from the distal end of the shaft through the one or more aspiration lumens; controls operation of the coblation energy source to selectively deliver coblation energy to the coblation tip; operates the fluid control apparatus such that the fluid is delivered to the distal end of the shaft at the same time as or for a selected period of time before coblation energy is delivered to the coblation tip; and operates the fluid control apparatus such that the fluid is delivered to the distal end of the shaft for a selected period of time after the delivery of coblation energy to the coblation tip has terminated.

In various embodiments, the coblation systems may include one or more of the following features: fluid control apparatus may include a temperature control mechanism; the coblation probe may be held at a fixed position relative to the shaft; the coblation probe may be movable within the shaft such that the coblation probe has a retracted position in which a tip of the coblation probe is proximate the distal end of the shaft and an extended position in which the tip of the coblation probe is spaced from the distal end of the shaft; etc.

In another aspect, a coblation device is provided that includes a shaft having a proximal end and distal end; one or more delivery lumens extending through the shaft from the proximal end towards the distal end; one or more aspiration lumens extending through the shaft from the proximal end towards the distal end; a coblation probe extending through the shaft, wherein the coblation probe includes a coblation tip proximate the distal end of the shaft; wherein the coblation probe is movable within the shaft such that the coblation probe has a retracted position in which a tip of the coblation probe is proximate the distal end of the shaft and an extended position in which the tip of the coblation probe is spaced from the distal end of the shaft.

In another aspect, a method of removing tissue is provided, the method including providing a coblation system having a shaft with a proximal end and distal end; one or more delivery lumens extending through the shaft from the proximal end towards the distal end; one or more aspiration lumens extending through the shaft from the proximal end towards the distal end; fluid control apparatus fluidly coupled to the one or more delivery lumens and the one or more aspiration lumens; a coblation probe extending through the shaft, wherein the coblation probe has a coblation tip proximate the distal end of the shaft; and a coblation energy source operatively coupled to the coblation probe to deliver coblation energy to the coblation tip. The method further includes positioning the distal end of the shaft proximate an ablation site using ultrasonic imaging; delivering fluid to the ablation site through the one or more delivery lumens; removing at least a portion of the fluid from the ablation site through the one or more aspiration lumens; and selectively delivering coblation energy to the coblation tip to remove tissue at the ablation site; wherein the fluid is delivered to the ablation site at the same time as or for a selected period of time before coblation energy is delivered to the coblation tip; and wherein the fluid is delivered to the ablation site for a selected period of time after the delivery of coblation energy to the coblation tip is terminated.

In various embodiments, the method may include one or more of the following features: delivering the fluid to the ablation site at a temperature below 20 degrees Celsius; the coblation probe may be movable within the shaft such that the coblation probe has a retracted position in which a tip of the coblation probe is proximate the distal end of the shaft and an extended position in which the tip of the coblation probe is spaced from the distal end of the shaft, and the method may include moving the coblation probe between the retracted and extended positions, and wherein moving the coblation probe between the retracted and extended positions may be performed while delivering fluid to the ablation site; etc.

In another aspect, a tissue dilation apparatus may be provided that includes an ultrasonically echogenic shaft having a proximal end and a distal end, wherein a longitudinal axis extends between the proximal end and the distal end, wherein the shaft includes a tube, and wherein the shaft has a shaft length extending along the longitudinal axis from the proximal end to the distal end; an expansion device proximate the distal end of the shaft, wherein the expansion device has a collapsed configuration and an expanded configuration, wherein the expansion device has a radial dimension relative to the longitudinal axis in each of the collapsed configuration and the expanded configuration, and wherein the radial dimension of the expansion device in the expanded configuration is larger than the radial dimension of the expansion device in the collapsed configuration; and an expansion lumen extending along the shaft from the proximal end towards the distal end, wherein the expansion lumen is in fluid communication with an interior of the expansion device.

In various embodiments, the tissue dilation apparatus may include one or more of the following features: a main channel may extend through the shaft between an opening at the proximal end to an opening at the distal end; the shaft may be a rigid shaft; the shaft may be a rigid metallic shaft; the radial dimension of the expansion device in the expanded configuration may be at least 1.5 times larger than the radial dimension of the expansion device in the collapsed configuration; the expansion device may be an inflatable balloon; the expansion device may extend along the shaft for at least 25% of the shaft length; the expansion lumen may be in fluid communication with an interior of the expansion device through one or more holes opening into the interior of the expansion device, wherein the expansion lumen extends through an interior of the shaft.

In another aspect, a coblation and tissue dilation kit is provided that includes a coblation device and a tissue dilation apparatus. The coblation device includes a coblation shaft having a proximal end and distal end; one or more delivery lumens extending through the coblation shaft from the proximal end towards the distal end; one or more aspiration lumens extending through the coblation shaft from the proximal end towards the distal end; and a coblation probe extending through the coblation shaft, wherein the coblation probe comprises a coblation tip proximate the distal end of the coblation shaft. The tissue dilation apparatus includes an ultrasonically echogenic dilation shaft having a proximal end and a distal end, wherein a longitudinal axis extends between the proximal end and the distal end, and wherein a main channel extends through the dilation shaft, the main channel sized to allow advancement of the coblation shaft of the coblation device from the proximal end to the distal end of the dilation shaft; an expansion device proximate the distal end of the dilation shaft, wherein the expansion device has a collapsed configuration and an expanded configuration, wherein the expansion device has a radial dimension relative to the longitudinal axis in each of the collapsed configuration and the expanded configuration, and wherein the radial dimension of the expansion device in the expanded configuration is larger than the radial dimension of the expansion device in the collapsed configuration; and an expansion lumen extending along the dilation shaft from the proximal end towards the distal end, wherein the expansion lumen is in fluid communication with an interior of the expansion device.

In various embodiments, the kits may include one or more of the following features: a coblation probe that is movable within the coblation shaft such that the coblation probe has a retracted position in which a tip of the coblation probe is proximate the distal end of the shaft and an extended position in which the tip of the coblation probe is spaced from the distal end of the shaft; fluid control apparatus fluidly coupled to the one or more delivery lumens and the one or more aspiration lumens; a coblation energy source operatively coupled to the coblation probe to deliver coblation energy to the coblation tip; a system controller operatively connected to the fluid control apparatus and the coblation energy source, wherein the system controller further controls operation of the fluid control apparatus such that a fluid is delivered to the distal end of the shaft through the one or more delivery lumens and that at least a portion of the fluid is removed from the distal end of the shaft through the one or more aspiration lumens; controls operation of the coblation energy source to selectively deliver coblation energy to the coblation tip; operates the fluid control apparatus such that the fluid is delivered to the distal end of the shaft at the same time as or for a selected period of time before coblation energy is delivered to the coblation tip; and operates the fluid control apparatus such that the fluid is delivered to the distal end of the shaft for a selected period of time after the delivery of coblation energy to the coblation tip has terminated; etc.

In another aspect, a coblation system with ultrasound artifact reduction may be provided. The system may include a shaft having a proximal end and distal end; one or more delivery lumens extending through the shaft from the proximal end towards the distal end; one or more aspiration lumens extending through the shaft from the proximal end towards the distal end; fluid control apparatus fluidly coupled to the one or more delivery lumens and the one or more aspiration lumens; a coblation probe extending through the shaft, wherein the coblation probe comprises a coblation tip proximate the distal end of the shaft; a coblation energy source operatively coupled to the coblation probe to deliver coblation energy to the coblation tip; a system controller operatively connected to the fluid control apparatus and the coblation energy source. The system controller controls operation of the fluid control apparatus such that a fluid is delivered to the distal end of the shaft through the one or more delivery lumens and that at least a portion of the fluid is removed from the distal end of the shaft through the one or more aspiration lumens; and the system controller also controls operation of the coblation energy source to selectively deliver coblation energy to the coblation tip; wherein the system controller operates the fluid control apparatus such that the fluid is delivered to the distal end of the shaft at the same time as or for a selected period of time before coblation energy is delivered to the coblation tip; and wherein the system controller operates the fluid control apparatus such that the fluid is delivered to the distal end of the shaft for a selected period of time after the delivery of coblation energy to the coblation tip has terminated. The fluid control apparatus may also include a temperature control mechanism that may optionally be operably connected to and controlled by the system controller.

In another aspect, a method of removing tissue using coblation may be provided. The method may include providing a coblation system that includes a shaft having a proximal end and distal end; one or more delivery lumens extending through the shaft from the proximal end towards the distal end; one or more aspiration lumens extending through the shaft from the proximal end towards the distal end; fluid control apparatus fluidly coupled to the one or more delivery lumens and the one or more aspiration lumens; a coblation probe extending through the shaft, wherein the coblation probe has a coblation tip proximate the distal end of the shaft; and a coblation energy source operatively coupled to the coblation probe to deliver coblation energy to the coblation tip. The method may further include positioning the distal end of the shaft proximate an ablation site using ultrasonic imaging; delivering fluid to the ablation site through the one or more delivery lumens; removing at least a portion of the fluid from the ablation site through the one or more aspiration lumens; and selectively delivering coblation energy to the coblation tip to remove tissue at the ablation site; wherein the fluid is delivered to the ablation site at the same time as or for a selected period of time before coblation energy is delivered to the coblation tip; and wherein the fluid is delivered to the ablation site for a selected period of time after the delivery of coblation energy to the coblation tip is terminated. The method may optionally include delivering the fluid to the ablation site at a temperature below 20 degrees Celsius.

In another aspect, a tissue dilation apparatus may be provided that includes an ultrasonically echogenic shaft having a proximal end and a distal end, wherein a longitudinal axis extends between the proximal end and the distal end, wherein the shaft is in the form of a hollow tube, and wherein the shaft has a shaft length extending along the longitudinal axis from the proximal end to the distal end; an expansion device proximate the distal end of the shaft, wherein the expansion device has a collapsed configuration and an expanded configuration, wherein the expansion device has a radial dimension relative to the longitudinal axis in each of the collapsed configuration and the expanded configuration, and wherein the radial dimension of the expansion device in the expanded configuration is at least 1.25 times (or in some instance at least 1.5 times) as large as the radial dimension of the expansion device in the collapsed configuration, wherein the expansion device optionally is in the form of an inflatable balloon, and further wherein the expansion device extends along the shaft for at least 25% of the shaft length; and a lumen extending along the shaft from the proximal end towards the distal end, wherein the lumen is in fluid communication with an interior of the expansion device through one or more holes opening into the interior of the expansion device, wherein the lumen optionally extends through an interior of the tube.

The details of one or more embodiments are set forth in the accompanying drawings and the description below. Other features and potential advantages will be apparent from the description and drawings, and from the claims.

### BRIEF DESCRIPTION OF DRAWINGS

FIGS. 1A & 1B depict the distal end of one embodiment of a coblation device which includes a coblation probe extending through a shaft.

FIGS. 2A & 2B depict an alternate embodiment of a coblation device including a coblation probe extending through a shaft.

FIG. 3 depicts a coblation probe movable between an extended position and a retracted position as depicted by the bidirectional arrows in FIG. 3.

FIG. 4 is a block diagram of one exemplary coblation system including a coblation device, fluid control apparatus, coblation energy source, and a system controller that may be operably connected to both the fluid control apparatus and the coblation energy source.

FIG. 5 is a side view of one tissue dilation apparatus with an expansion device in the collapsed configuration.

FIG. 6 is a side view of the tissue dilation apparatus of FIG. 5 with the expansion device in an expanded configuration.

FIG. 7 is an end view taken along the longitudinal axis of the shaft of the tissue dilation apparatus of FIG. 6.

FIG. 8 is a cross-sectional view of another exemplary embodiment of a tissue dilation apparatus that may be used with the devices and systems described herein.

Like reference symbols in the various drawings indicate like elements.

### DETAILED DESCRIPTION OF ILLUSTRATIVE EMBODIMENTS

In the following detailed description of illustrative embodiments, reference is made to the accompanying figures of the drawing which form a part hereof, and in which are shown, by way of illustration, specific embodiments of this disclosure. It is to be understood that other embodiments may be utilized and structural changes may be made without departing from the scope of the present disclosure.

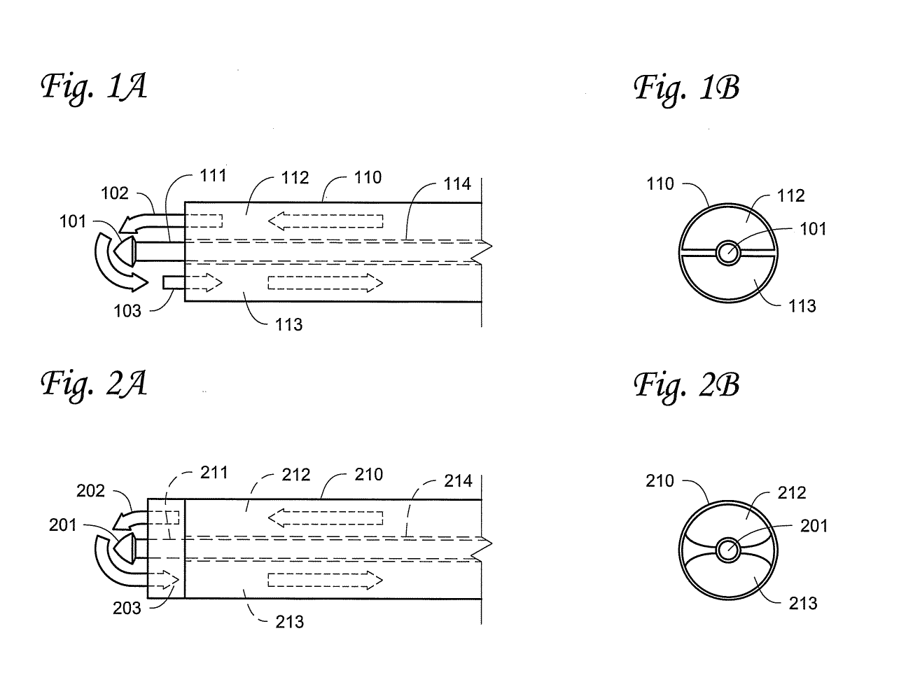

FIGS. 1A & 1B depict the distal end of one embodiment of a coblation device which includes a coblation probe 111 that extends through the shaft 110. The shaft 110 also incorporates two or more lumens 112 and 113. Of the two or more lumens, one or more lumens will be used to deliver fluid to the ablation site (referred to as a delivery lumen) and one or more of the lumens will be used to remove fluid from the ablation site (referred to as an aspiration lumen). As depicted in FIG. 1A, lumen 112 operates as the delivery lumen to deliver fluid as depicted by arrow 102, while lumen 113 operates as the aspiration lumen to remove fluid and/or tissue from the ablation site as depicted by arrow 103.

The channel 114 in the shaft 110, within which the coblation probe 111 is located, may preferably extend to the distal end of the shaft 110 as depicted in FIG. 1A. The coblation probe 111 may extend past the distal end of the shaft 110 such that the tip 101 of the coblation probe 111 is spaced from the distal end of the shaft as seen in FIG. 1A.

FIGS. 2A & 2B depict an alternate embodiment of a coblation device including a coblation probe 211 that extends through the shaft 210. The shaft 210 also incorporates two or more lumens 212 and 213. Of the two or more lumens, one or more lumens will be used to deliver fluid to the ablation site (referred to as a delivery lumen) and one or more of the lumens will be used to remove fluid from the ablation site (referred to as an aspiration lumen). As depicted in FIG. 2A, lumen 212 operates as the delivery lumen to deliver fluid as depicted by arrow 202, while lumen 213 operates as the aspiration lumen to remove fluid from the ablation site as depicted by arrow 203.

The channel 214 in the shaft 210, from which the coblation probe 211 extends, may preferably terminate short of the distal end of the shaft 210 as depicted in FIG. 2A. As a result, the outer wall of the shaft 210 may form a shroud over a portion of the coblation probe 211. The tip 201 of the coblation probe 211 may still preferably be located past the distal end of the shaft 210 as depicted in FIG. 2A.

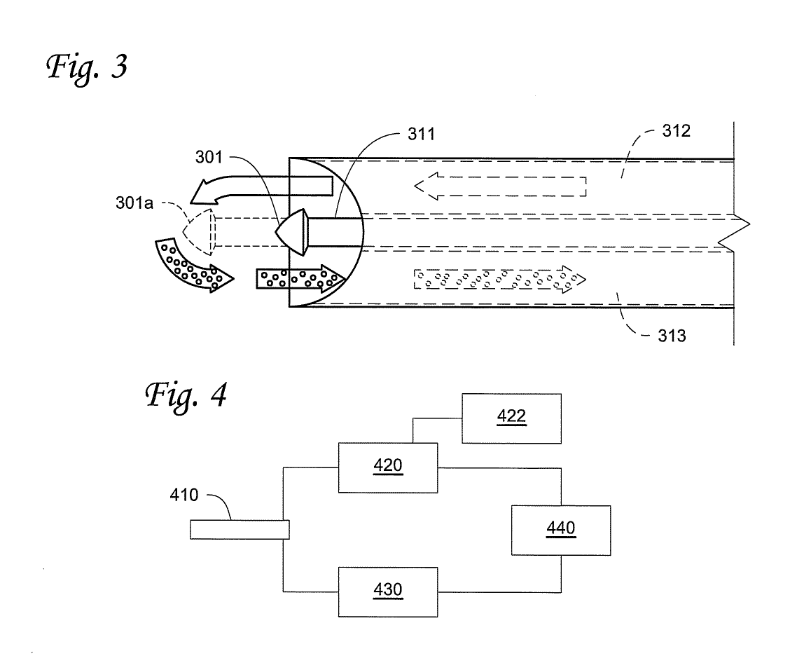

Although the coblation probes of coblation systems described herein may have stationary positions relative to the shafts in which they are located such that the coblation probe is held at a fixed position relative to the shaft, in some embodiments, the coblation probe may be capable of moving relative to the shaft. FIG. 3 depicts one embodiment of this optional feature, with the coblation probe 311 being movable between an extended position and a retracted position as depicted by the bidirectional arrows in FIG. 3. In the extended position, the tip of the probe 311 is advanced in the distal direction out of the shaft 310 (see shaded portion of figure and reference number 301a) such that the tip of the probe is spaced from the distal end of the shaft 310. In the retracted position as depicted in FIG. 3, the tip 301 of the probe 311 is closer to the distal end of the shaft 310, and may, in some embodiments, be withdrawn into the shaft 310.

Withdrawal of the probe 311 from the extended position towards the proximal end of the shaft 310 may preferably create a void that will be filled by the fluid delivered through the delivery lumen. This movement and subsequent filling may enhance aspiration through lumen 313 of the fluid delivered to the ablation site through lumen 312.

FIG. 4 is a block diagram of one exemplary coblation system, wherein the system includes a coblation device 410, fluid control apparatus 420 that may be fluidly connected to the coblation device 410, coblation energy source 430 that may be operably connected to a coblation probe (not shown) provided as a part of the coblation device 410, and a system controller 440 that may be operably connected to both the fluid control apparatus 420 and the coblation energy source 430.

The fluid control apparatus 420 may optionally include a temperature control mechanism 422 capable of controlling the temperature of the fluid delivered to the coblation device 410 from the fluid control apparatus 420. The temperature control mechanism may be in the form of a refrigeration unit or a thermal sink capable of reducing the temperature of the fluid delivered to the coblation device to a temperature below 20 degrees Celsius (or any other selected temperature).

In addition to the coblation systems described herein, the present disclosure also encompasses tissue dilation apparatus that may potentially be used to provide access for a coblation probe and/or may be used alone to provide tissue dilation.

In one exemplary embodiment, the tissue dilation apparatus may include a shaft with a pointed or tapered tip at one end a fluid connection fitting at the opposite end. An expansion device (in the form of, e.g., an inflatable balloon) may preferably be attached to the shaft proximate the distal end of the shaft.

The shaft may be hollow such that fluids can be delivered through the shaft as discussed herein and/or the shaft may be sized to operate as a cannula with one or more lumens that can be used to deliver one or more working instruments, fluids, etc. to targeted body tissue. For example, the shaft may be used to deliver a coblation probe.

The shaft may be of any desired thickness, although it may be preferred that the shaft be in the range of 10 gauge to 30 gauge. It may be preferred that the shaft be substantially rigid such that it does not deflect significantly under normal use (e.g., the shaft may be made of metals or other materials capable of providing the desired rigidity).

Although stainless steel may preferably be used to construct the shaft, many other materials could be substituted. In general, however, it may be preferred that at least some material used in the shaft be ultrasonically echogenic where echogenic materials are materials may be visualized or otherwise guided using ultrasonic energy. In some instances, the shaft may be, e.g., coated or otherwise treated (e.g., etched, etc.) to improve its ability to be ultrasonically detected.

The shaft may, in some embodiments, have a solid tip (as opposed to the open tips of, e.g., Quincke-type needles) and the lumen defined in the hollow shaft may terminate at one or more openings through the sidewall of the shaft. Those openings may preferably be in fluid communication with the interior of an inflatable balloon attached to the shaft. The openings may, in some instances, improve the echogenicity to improve conspicuity of the apparatus when using ultrasonic energy. The use of multiple openings into the interior of the expansion device may be useful for more accurately determining the location of the expansion device.

The proximal end of the shaft may include a fluid connection such as, e.g., a Luer lock fitting, etc. to permit fluid-tight attachment to a source of fluid to be delivered through the lumen of the shaft. In use, the fitting may be attached to a syringe or other fluid delivery device. In embodiments to be used an access cannula to deliver one or more working instruments to targeted body tissue, the proximal end of the shaft may include one or more connectors that connect to, e.g., a working instrument, coblation probe, etc. as described herein.

The length of the shaft may vary depending on the needs of the user, although in some embodiments the length of the shaft may be, e.g., 200 millimeters (mm) or less (where the shaft length is measured from the proximal end to the distal end). In some embodiments, the length may preferably be 100 mm or less. It may be preferred that the expansion device (e.g., inflatable balloon) occupy a distance of about 10% or more of the length of the shaft. In other embodiments, the expansion device may occupy 25% or more of the length of the shaft. In terms of actual dimensions, the expansion device may have a length of, e.g., 5 mm or more, 10 mm or more, 20 mm or more, or even 50 mm or more.

The expansion device located near the distal end of the apparatus may be in the form of one or more inflatable balloons. Materials used for the balloons may preferably be flexible such that the expansion device is capable of taking a collapsed configuration for delivery to a desired location where the expansion device can be expended to an expanded configuration. Suitable materials may include, e.g., polyethylenes, etc. It may be preferred that the balloon material allow for the transmission of ultrasonic energy such that the portion of the shaft located within the balloon can be detected using ultrasonic energy.

The expansion device may preferably have a profile or shape that is relatively small, e.g., similar to the dimensions of the shaft on or to which the expansion device is mounted. The expansion device may take on a variety of shapes when in the expanded configuration. Examples of some potential shapes in the expanded configuration may include, but are not limited to narrow oblong shapes, near spherical shapes, spherical shapes, two or more lobes, etc.

When deploying the tissue dilation apparatus, ultrasound guidance may be used to guide the shaft to a desired internal body location with the expansion device is preferably in the collapsed configuration. This process may be similar to that used in an ultrasound-guided injection and/or other procedures as described herein. The shaft may, for example, be passed through the area in which the stenosis is located until the expansion device (e.g., balloon) is positioned in the region to be dilated.

With the expansion device in position, the expansion device is then expanded from the collapsed configuration to the expanded configuration. When the expansion device is in the form of an inflatable balloon, the expansion may preferably be caused by passing a fluid such as, e.g., distilled sterile water, de-gassed sterile ultrasound gel, etc. It may be preferred that the fluid used be non-echogenic such that ultrasonic visualization of the expansion site is not significantly impaired during the expansion process. It may further preferred that the distention caused by the expansion device be monitored (e.g., visualized) using concomitant ultrasound imaging or any other suitable visualization technology. The expansion device may be maintained in its expanded configuration for any selected period of time, e.g., from a few seconds to minutes. Further, the expansion device may be expanded and collapsed only once or it may be repeatedly expanded and collapsed as need to provide the desired tissue distention. If the expansion device is repeatedly expanded and collapsed, the amount of expansion may be varied, e.g., the size of the expansion device may be progressively increased with each expansion, etc.

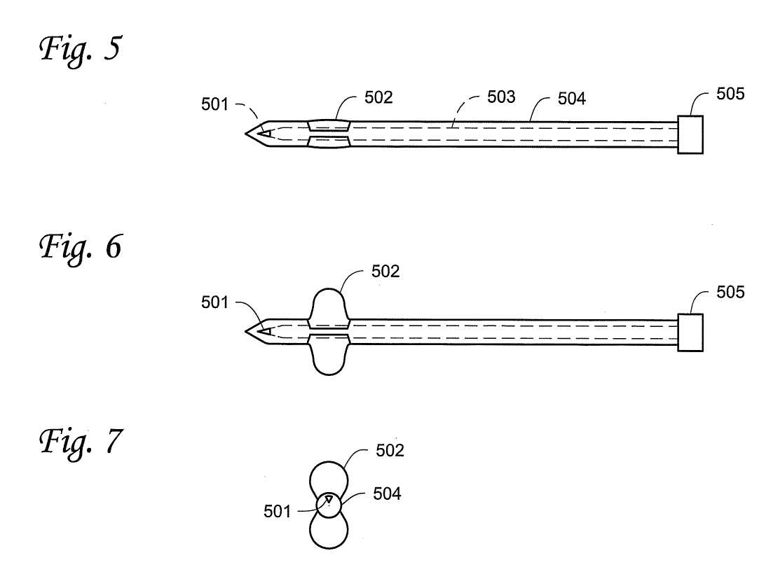

FIG. 5 is a side view of one illustrative embodiment of a tissue dilation apparatus 510, with the expansion device 502 in the collapsed configuration. FIG. 6 is a side view of the tissue dilation apparatus 510 of FIG. 5 with the expansion device 502 in an expanded configuration. FIG. 7 is an end view taken along the longitudinal axis of the shaft of the tissue dilation apparatus of FIG. 5.

The tissue dilation apparatus 510 of FIGS. 5-7 includes a shaft 504 that has a lumen 503 extending therethrough. The shaft 504 includes an optional beveled opening 501 at its distal end that may preferably be closed or sealed to permit expansion of the expansion device 502. The shaft 504 may also include a fitting 505 at its proximal end to facilitate attachment of a fluid delivery device (e.g., syringe, pump, etc.) or other connector to adapt to a working instrument as discussed herein.

Although both lobes of the expansion device 502 are expanded (as depicted in FIGS. 6-7), in some embodiments, the different lobes may be selectively expandable (by, e.g., delivering fluids thought separate lumens, etc.). Also, although the expansion device 502 is depicted as being contained within the shaft 504 in the collapsed configuration, the expansion device 502 may alternatively be wrapped around the exterior of the shaft 504 (in a manner similar to cardiac balloon dilators).

Although the expansion device 502 may be filled by delivering a fluid through the main channel of the shaft 504 (where, e.g., the distal end of the shaft 504 is sealed), an auxiliary channel located within the shaft 504 (if hollow) or positioned external to the shaft 504 (whether the shaft 504 is solid or hollow) may be used to deliver fluids to and/or remove fluids from the expansion device 502. If the shaft 504 is hollow and an auxiliary channel is used to dilate the expansion device 502, the hollow shaft 504 can potentially be used to deliver other fluids (e.g., therapeutic substances, etc.) or other devices such as the coblation probes described herein to an internal body location.

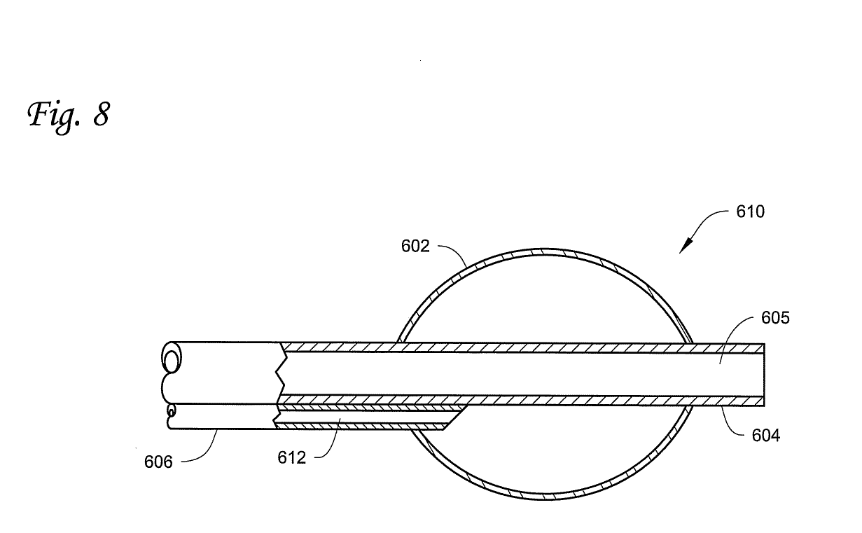

FIG. 8 is a cross-sectional view of another exemplary embodiment of a tissue dilation apparatus 610 in which the expansion device 602 is in the form of a hollow balloon located on the exterior of a primary shaft 604. The expansion device 602 is in fluid communication with a lumen 612 passing through an auxiliary shaft 606 that is separate from the main channel 605 extending through the primary shaft 604. As a result, fluids can be delivered to and/or removed from the expansion device 602 through the lumen 612. Although the auxiliary shaft 606 is depicted as attached to the primary shaft 604, this attachment is optional.

With the expansion device 602 in a selected position, the expansion device 602 can be dilated to the expanded configuration (as depicted in FIG. 8). The main channel 605 of the primary shaft 604 can then be used to introduce other devices (e.g., coblation probes, etc.), fluids (e.g., therapeutic substances, etc.) to a location proximate the dilated expansion device 602.

In some instances, the primary shaft 604 may be flexible, with the primary shaft 604 and collapsed expansion device 602 being introduced to a selected body site while a support structure (e.g., a solid or hollow needle, etc.) is located within the main channel 605 (to give the primary shaft 604 sufficient stiffness to facilitate advancement to a selected location). After the expansion device 602 is in the selected position (and possibly dilated), the support structure may be removed from the main channel 605, with the primary shaft 604 and its main channel 605 serving as a defined path through which other devices, fluids, etc. can be delivered. For example, the main channel 605 may be sized to allow a coblation device (such as, e.g., the coblation devices described herein) to be advanced through the main channel 605.

Although the primary shaft 604 may itself be echogenic, in some embodiments, the echogenicity may be provided by the support structure (if used) while the primary shaft 604 and the expansion device 602 are substantially transparent to the ultrasonic energy used for guiding the tissue dilation apparatus. In such an embodiment, the echogenic support structure may be removed to allow unobstructed ultrasonic visualization of the body site at which the expansion device 602 is deployed (in those embodiments in which the primary shaft 604 and the expansion device 602 are non-echogenic). In still another variation, the auxiliary shaft 606 may be echogenic while the primary shaft 606 is not, such that ultrasonic guidance can be used to position the expansion device 602, with the primary shaft 604 obscuring less of the field of interest.

Also, in some embodiments, a pressure sensor may be provided to monitor fluid pressure within the expansion device.

The systems, apparatus and methods described herein may advantageously be used in connection with the systems, devices and methods described in, e.g., PCT Patent Application No. PCT/US09/34659, titled SYSTEMS, DEVICES AND METHODS FOR ACCESSING BODY TISSUE, filed on even date herewith and/or U.S. Provisional Patent Application Ser. No. 61/030,009, titled ACCESSING AND TREATING BODY TISSUE, filed on Feb. 20, 2008.

The words “preferred” and “preferably” as used herein refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, a coblation probe may refer to one, two or more coblation probes.

The term “and/or” means one or all of the listed elements or a combination of any two or more of the listed elements.

The complete disclosure of the patents, patent documents, and publications identified herein are incorporated by reference in their entirety as if each were individually incorporated.

Illustrative embodiments are discussed and reference has been made to possible variations within the scope of this disclosure. These and other variations and modifications will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A coblation system with ultrasound artifact reduction, the system comprising:
a shaft comprising a proximal end and distal end;
one or more delivery lumens extending through the shaft from the proximal end towards the distal end;
one or more aspiration lumens extending through the shaft from the proximal end towards the distal end;
fluid control apparatus fluidly coupled to the one or more delivery lumens and the one or more aspiration lumens;
a coblation probe extending through the shaft, wherein the coblation probe comprises a coblation tip proximate the distal end of the shaft;
a coblation energy source operatively coupled to the coblation probe to deliver coblation energy to the coblation tip;
a system controller operatively connected to the fluid control apparatus and the coblation energy source, wherein the system controller further:
controls operation of the fluid control apparatus such that a fluid is delivered to the distal end of the shaft through the one or more delivery lumens and that at least a portion of the fluid is removed from the distal end of the shaft through the one or more aspiration lumens;
controls operation of the coblation energy source to selectively deliver coblation energy to the coblation tip;
wherein the system controller operates the fluid control apparatus such that the fluid is delivered to the distal end of the shaft at the same time as or for a selected period of time before coblation energy is delivered to the coblation tip;
and wherein the system controller operates the fluid control apparatus such that the fluid is delivered to the distal end of the shaft for a selected period of time after the delivery of coblation energy to the coblation tip has terminated.

2. A system according to claim 1, wherein the fluid control apparatus further comprises a temperature control mechanism.

3. A system according to claim 1, wherein the coblation probe is held at a fixed position relative to the shaft.

4. A system according to claim 1, wherein the coblation probe is movable within the shaft such that the coblation probe comprises a retracted position in which a tip of the coblation probe is proximate the distal end of the shaft and an extended position in which the tip of the coblation probe is spaced from the distal end of the shaft.

5. A coblation device comprising:
a shaft comprising a proximal end and distal end;
one or more delivery lumens extending through the shaft from the proximal end towards the distal end;
one or more aspiration lumens extending through the shaft from the proximal end towards the distal end;
a coblation probe extending through the shaft, wherein the coblation probe comprises a coblation tip proximate the distal end of the shaft;
wherein the coblation probe is movable within the shaft such that the coblation probe comprises a retracted position in which a tip of the coblation probe is proximate the distal end of the shaft and an extended position in which the tip of the coblation probe is spaced from the distal end of the shaft.

6. A method of removing tissue, the method comprising:
providing a coblation system comprising:
a shaft comprising a proximal end and distal end;
one or more delivery lumens extending through the shaft from the proximal end towards the distal end;
one or more aspiration lumens extending through the shaft from the proximal end towards the distal end;
fluid control apparatus fluidly coupled to the one or more delivery lumens and the one or more aspiration lumens;
a coblation probe extending through the shaft, wherein the coblation probe comprises a coblation tip proximate the distal end of the shaft;
a coblation energy source operatively coupled to the coblation probe to deliver coblation energy to the coblation tip;
positioning the distal end of the shaft proximate an ablation site using ultrasonic imaging;
delivering fluid to the ablation site through the one or more delivery lumens;
removing at least a portion of the fluid from the ablation site through the one or more aspiration lumens; and
selectively delivering coblation energy to the coblation tip to remove tissue at the ablation site;
wherein the fluid is delivered to the ablation site at the same time as or for a selected period of time before coblation energy is delivered to the coblation tip;
and wherein the fluid is delivered to the ablation site for a selected period of time after the delivery of coblation energy to the coblation tip is terminated.

7. A method according to claim 6, further comprising delivering the fluid to the ablation site at a temperature below 20 degrees Celsius.

8. A method according to claim 6, wherein the coblation probe is movable within the shaft such that the coblation probe comprises a retracted position in which a tip of the coblation probe is proximate the distal end of the shaft and an extended position in which the tip of the coblation probe is spaced from the distal end of the shaft. and wherein the method comprises moving the coblation probe between the retracted and extended positions.

9. A method according to claim 8, wherein moving the coblation probe between the retracted and extended positions is performed while delivering fluid to the ablation site.

10. A tissue dilation apparatus comprising:
an ultrasonically echogenic shaft comprising a proximal end and a distal end, wherein a longitudinal axis extends between the proximal end and the distal end, wherein the shaft comprises a tube, and wherein the shaft comprises a shaft length extending along the longitudinal axis from the proximal end to the distal end;
an expansion device proximate the distal end of the shaft, wherein the expansion device comprises a collapsed configuration and an expanded configuration, wherein the expansion device comprises a radial dimension relative to the longitudinal axis in each of the collapsed configuration and the expanded configuration, and wherein the radial dimension of the expansion device in the expanded configuration is larger than the radial dimension of the expansion device in the collapsed configuration; and
an expansion lumen extending along the shaft from the proximal end towards the distal end, wherein the expansion lumen is in fluid communication with an interior of the expansion device.

11. An apparatus according to claim 10, further comprising a main channel extending through the shaft between an opening at the proximal end to an opening at the distal end.

12. An apparatus according to claim 10, wherein the shaft comprises a rigid shaft.

13. An apparatus according to claim 10, wherein the shaft comprises a rigid metallic shaft.

14. An apparatus according to claim 10, wherein the radial dimension of the expansion device in the expanded configuration is at least 1.5 times larger than the radial dimension of the expansion device in the collapsed configuration.

15. An apparatus according to claim 10, wherein the expansion device comprises inflatable balloon.

16. An apparatus according to claim 10, wherein the expansion device extends along the shaft for at least 25% of the shaft length.

17. An apparatus according to claim 10, wherein the expansion lumen is in fluid communication with an interior of the expansion device through one or more holes opening into the interior of the expansion device, wherein the expansion lumen extends through an interior of the shaft.

18. A coblation and tissue dilation kit comprising a coblation device and a tissue dilation apparatus;
wherein the coblation device comprises:
a coblation shaft comprising a proximal end and distal end;
one or more delivery lumens extending through the coblation shaft from the proximal end towards the distal end;
one or more aspiration lumens extending through the coblation shaft from the proximal end towards the distal end; and
a coblation probe extending through the coblation shaft, wherein the coblation probe comprises a coblation tip proximate the distal end of the coblation shaft;
and wherein the tissue dilation apparatus comprises:
an ultrasonically echogenic shaft comprising a proximal end and a distal end, wherein a longitudinal axis extends between the proximal end and the distal end, and wherein a main channel extends through the shaft, the main channel sized to allow advancement of the coblation shaft of the coblation device from the proximal end to the distal end of the ultrasonically echogenic dilation shaft;
an expansion device proximate the distal end of the dilation shaft, wherein the expansion device comprises a collapsed configuration and an expanded configuration, wherein the expansion device comprises a radial dimension relative to the longitudinal axis in each of the collapsed configuration and the expanded configuration, and wherein the radial dimension of the expansion device in the expanded configuration is larger than the radial dimension of the expansion device in the collapsed configuration; and
an expansion lumen extending along the dilation shaft from the proximal end towards the distal end, wherein the expansion lumen is in fluid communication with an interior of the expansion device.

19. A kit according to claim 18, the kit further comprising:
fluid control apparatus fluidly coupled to the one or more delivery lumens and the one or more aspiration lumens;
a coblation energy source operatively coupled to the coblation probe to deliver coblation energy to the coblation tip; and
a system controller operatively connected to the fluid control apparatus and the coblation energy source, wherein the system controller further:
controls operation of the fluid control apparatus such that a fluid is delivered to the distal end of the shaft through the one or more delivery lumens and that at least a portion of the fluid is removed from the distal end of the shaft through the one or more aspiration lumens;
controls operation of the coblation energy source to selectively deliver coblation energy to the coblation tip;
operates the fluid control apparatus such that the fluid is delivered to the distal end of the shaft at the same time as or for a selected period of time before coblation energy is delivered to the coblation tip;
and operates the fluid control apparatus such that the fluid is delivered to the distal end of the shaft for a selected period of time after the delivery of coblation energy to the coblation tip has terminated.

20. A kit according to claim 18, wherein the coblation probe is movable within the coblation shaft such that the coblation probe comprises a retracted position in which a tip of the coblation probe is proximate the distal end of the shaft and an extended position in which the tip of the coblation probe is spaced from the distal end of the shaft.
